(19) United States
(12) Patent Application Publication
(10) Pub. No.: US 2021/0220662 A1
(43) Pub. Date: Jul. 22, 2021

(54) Cold Plasma Therapy Device with Enhanced Safety

(71) Applicants: Dan Liu, Hockessin, DE (US); Sean Xiaolu Wang, Wilmington, DE (US)

(72) Inventors: Dan Liu, Hockessin, DE (US); Sean Xiaolu Wang, Wilmington, DE (US)

(73) Assignee: B&W Tek, Inc., Newark, DE (US)

(21) Appl. No.: 17/143,442

(22) Filed: Jan. 7, 2021

Related U.S. Application Data
(60) Provisional application No. 62/961,909, filed on Jan. 16, 2020.

(51) Int. Cl. A61N 1/44; H05H 1/46; H05H 1/24

(52) U.S. Cl. CPC A61N 1/44; H05H 2001/4695; H05H 1/2406; H05H 1/46

(57) ABSTRACT

This invention discloses a cold plasma therapy device with enhanced safety. The plasma therapy device comprises a dielectric barrier made of a material with high dielectric constant (i.e., relative permittivity). Hence the thickness of the dielectric barrier can be increased to produce similar plasma intensity in comparison to a dielectric barrier with a low dielectric constant. The increased thickness enhances the mechanical durability of the dielectric barrier. When the thickness of the dielectric barrier is larger than the maximum discharge gap of the ambient air (or the supplied gas medium) under the voltage applied, no arc discharge will be produced even when there is a crack across the thickness of the dielectric barrier. This minimizes the risk of subject tissue damage from possible electric shocks.

### REFERENCE TO RELATED APPLICATION

This application claims the inventions which were disclosed in Provisional patent Application No. 62/961,909, filed Jan. 16, 2020, entitled “COLD PLASMA THERAPY DEVICE WITH ENHANCED SAFETY.” The benefit under 35 USC § 119(e) of the above-mentioned United States Provisional Applications is hereby claimed, and the aforementioned application is hereby incorporated herein by reference.

### FIELD OF THE INVENTION

This invention generally relates to a plasma therapy device, and more specifically, to a cold plasma therapy device with enhanced safety.

### BACKGROUND

Plasma, as the fourth fundamental state of matter, is a neutral ionized gas composed of positively charged ions, electrons, and neutral particles. In typical thermal plasma, all particles approach thermal equilibrium due to intensive collisions between electrons and heavy particles. The temperature in such plasma can reach several thousand degrees. On the other hand, there is another type of plasma in which electrons and heavy particles are in thermal non-equilibrium. In this case, the temperature of the heavy particles is much lower than that of the electrons. This type of plasma is called non-thermal plasma or cold plasma. The heavy particle temperature in cold plasma is typically between 25° C. and 45° C. The plasma discharge may take place in ambient air or in specially supplied gas flow. Many reactive species, including oxygen-based radicals, nitrogen-based radicals, and other components, are generated in the cold plasma. This complicated chemistry can lead to various interactions between cold plasma and biological tissues, allowing the cold plasma to be used for biomedicine.

Dielectric barrier discharge (DBD), which involves electrical discharge between two electrodes separated by an insulating dielectric barrier, is one effective method to produce cold plasma. For biomedical applications, the living tissue is often employed as one of the electrodes, and the plasma discharge is produced between the dielectric barrier and the subject tissue. In general, a high voltage in the range from several kV (kilovolt) to a few tens of kV is supplied to the electrode to produce the plasma discharge. Under continued usage, the dielectric barrier may be damaged by mechanical, thermal, or other reasons or even by the plasma discharge and develop cracks in it. Such cracks can lead to arc discharge directly from the electrode to the subject tissue, which may cause severe tissue damage.

### SUMMARY OF THE INVENTION

It is the overall goal of the present invention to solve the above-mentioned problems and provide a cold plasma therapy device with enhanced safety. The plasma therapy device comprises a dielectric barrier made of a material with high dielectric constant (i.e., relative permittivity). Hence the thickness of the dielectric barrier can be increased to produce similar plasma intensity in comparison to a dielectric barrier with a low dielectric constant. The increased thickness enhances the mechanical durability of the dielectric barrier. When the thickness of the dielectric barrier is larger than the maximum discharge gap of the ambient air (or the supplied gas medium) under the voltage applied, no arc discharge will be produced even when there is a crack across the thickness of the dielectric barrier. This minimizes the risk of subject tissue damage from possible electric shocks.

### BRIEF DESCRIPTION OF THE FIGURES

The accompanying FIGURES, where like reference numerals refer to identical or functionally similar elements throughout the separate views and which together with the detailed description below are incorporated in and form part of the specification, serve to further illustrate various embodiments and to explain various principles and advantages all in accordance with the present invention.

FIG. 1 illustrates one exemplary embodiment of the DBD device.

Skilled artisans will appreciate that elements in the figures are illustrated for simplicity and clarity and have not necessarily been drawn to scale. For example, the dimensions of some of the elements in the FIGURES may be exaggerated relative to other elements to help to improve understanding of embodiments of the present invention.

### DETAILED DESCRIPTION

Before describing in detail embodiments that are in accordance with the present invention, it should be observed that the embodiments reside primarily in combinations of method steps and apparatus components related to a cold plasma therapy device with enhanced safety. Accordingly, the apparatus components and method steps have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

In this document, relational terms such as first and second, top and bottom, and the like may be used solely to distinguish one entity or action from another entity or action without necessarily requiring or implying any actual such relationship or order between such entities or actions. The terms “comprises,” “comprising,” or any other variation thereof are intended to cover a non-exclusive inclusion, such that a process, method, article, or apparatus that comprises a list of elements does not include only those elements but may include other elements not expressly listed or inherent to such process, method, article, or apparatus. An element proceeded by “comprises . . . a” does not, without more constraints, preclude the existence of additional identical elements in the process, method, article, or apparatus that comprises the element.

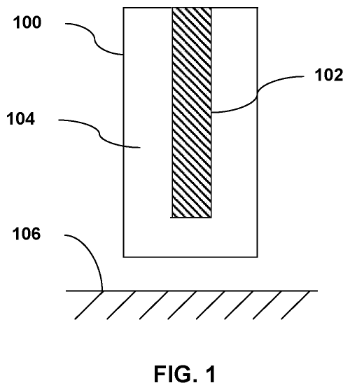

In one exemplary embodiment of the present invention, the cold plasma therapy device comprises a dielectric barrier discharge (DBD) probe 100 as shown in FIG. 1, which connects to a high voltage power supply through a high voltage cable (both not shown). The power supply is preferably a pulsed power supply with an adjustable repetition rate and output voltage. The output voltage is preferably in the range from 1 kV (kilovolt) to several tens of kV or even higher. The DBD probe 100 comprises an electrode 102 enclosed in a close-ended tube 104, which serves the dielectric barrier. The high voltage from the power supply produces a cold plasma discharge in the ambient air or in a supplied gas medium between the dielectric barrier 104 (i.e., the close-ended tube) and the subject tissue 106. Electrode 102 is made of an electrically conductive material such as a metal (e.g., copper) connected to the high voltage cable. The close-ended tube 104 can be cylindrical shaped or in other shapes suitable to be applied to the subject tissue 106. The end of tube 104 can be flat, spherical, or in different shapes depending on the application conditions. The close-ended tube 104 is made of a material with a high dielectric constant (i.e., relative permittivity). Preferably, the dielectric constant of the material is >5 and more preferably >10 or even >20. One example of such material is zirconium oxide (zirconia), which has a dielectric constant of >25. In comparison to the material with a relatively low dielectric constant (e.g., quartz, which has a dielectric constant of 3.8), the zirconia-based dielectric barrier can be made much thicker to produce similar plasma intensity under the same applied voltage since the capacitance of the dielectric barrier is proportional to εᵣ/d, where εᵣ is the dielectric constant and d is the thickness of the dielectric barrier, respectively. The increased thickness enhances the mechanical durability of the dielectric barrier. In an exemplary embodiment, the thickness of the zirconia-based dielectric barrier is larger than the maximum discharge gap of the ambient air (or the supplied gas medium) under the voltage applied such that no arc discharge will be produced even when there is a crack across the thickness of the dielectric barrier. As one example, the ambient air has a breakdown voltage of 3 kV/mm at standard atmospheric pressure. When the thickness of the zirconia-based dielectric barrier is made greater than 6 mm, no arc discharge will be produced between the electrode and the subject tissue under a supplied high voltage of 18 kV even in case the dielectric barrier is cracked. This minimizes the risk of subject tissue damage from possible electric shocks. In a slight variation of the present embodiment, the dielectric barrier of the DBD probe is made of a composite material comprising two or more layers of materials with high dielectric constants to further reduce the risk of crack induced arc discharge.

In the foregoing specification, specific embodiments of the present invention have been described. However, one of ordinary skill in the art appreciates that various modifications and changes can be made without departing from the scope of the present invention as set forth in the claims below. The numerical values cited in the specific embodiment are illustrative rather than limiting. Accordingly, the specification and FIGURES are to be regarded in an illustrative rather than a restrictive sense, and all such modifications are intended to be included within the scope of the present invention. The benefits, advantages, solutions to problems, and any element(s) that may cause any benefit, advantage, or solution to occur or become more pronounced are not to be construed as critical, required, or essential features or elements of any or all the claims. The invention is defined solely by the appended claims, including any amendments made during the pendency of this application and all equivalents of those claims as issued.

What is claimed is:

1. A dielectric barrier discharge (DBD) cold plasma therapy device for treating a subject, the DBD cold plasma therapy device comprising:
an electrode;
a dielectric barrier enclosing the electrode; and
a high voltage power supply applying a high voltage to the electrode to produce a cold plasma discharge in the ambient air or in a supplied gas medium between the dielectric barrier and the subject for treating the subject;
wherein the dielectric barrier is made of a material with a high dielectric constant of >5.

2. The DBD cold plasma therapy device of claim 1, wherein the dielectric barrier is made of a material with a high dielectric constant of >10.

3. The DBD cold plasma therapy device of claim 1, wherein the dielectric barrier is made of a material with a high dielectric constant of >20.

4. The DBD cold plasma therapy device of claim 1, wherein the dielectric barrier is made of zirconium oxide.

5. The DBD cold plasma therapy device of claim 1, wherein the thickness of the dielectric barrier is larger than the maximum discharge gap of the ambient air or the supplied gas medium under the applied high voltage.

6. The DBD cold plasma therapy device of claim 1, wherein the material is a composite material comprising two or more layers of materials with high dielectric constants.

7. The DBD cold plasma therapy device of claim 1, wherein the high voltage power supply is a pulsed, high voltage power supply with adjustable output voltage and repetition rate.
